(19) United States
(12) Patent Application Publication
(10) Pub. No.: US 2022/0059242 A1
(43) Pub. Date: Feb. 24, 2022

(54) METHOD AND SYSTEM FOR MANAGING EPIDEMICS THROUGH BAYESIAN LEARNING ON CONTACT NETWORKS

(71) Applicant: CALIFORNIA INSTITUTE OF TECHNOLOGY, Pasadena, CA (US)

(72) Inventors: Tapio Schneider, Pasadena, CA (US); Chiara Daraio, South Pasadena, CA (US); Oliver R. Dunbar, Pasadena, CA (US)

(21) Appl. No.: 17/445,426

(22) Filed: Aug. 19, 2021

Related U.S. Application Data
(60) Provisional application No. 63/068,097, filed on Aug. 20, 2020.

(51) Int. Cl. G16H 50/80; H04W 4/38; H04W 4/029; G16H 10/65; G16H 50/30

(52) U.S. Cl. CPC G16H 50/80; H04W 4/38; G16H 50/30; G16H 10/65; H04W 4/029

(57) ABSTRACT

Systems and methods for epidemic/pandemic management by creating a network of user devices wirelessly connected to a central server or servers, the devices having location/proximity capability. The central server propagates crowdsourced information about individual risks of exposure and infectiousness across a dynamic contact network, where the risk assessments are determined by the server by data assimilation methods with corrections made by updating previous risk network models and re-evolving them.

### CROSS-REFERENCE TO RELATED APPLICATIONS

The present application is related to U.S. Patent Application No. 63/068,097 filed on Aug. 20, 2020, the disclosure of which is incorporated herein by reference in its entirety.

### BACKGROUND

Local and global epidemics, such as COVID-19, are fought with non-pharmaceutical interventions (NPIs), including social distancing, mask usage, and restrictions of mass gatherings. However, some NPIs such as lockdowns come at catastrophic costs to individuals, economies, and societies, with disproportionate burdens carried by disadvantaged groups. Even if imposed only intermittently and regionally, lockdowns are an inefficient means of epidemic control: they isolate much of the population, although even at extreme epidemic peaks, only a few percent of the population are infectious. If individuals who are at high risk of being infectious could be identified before they infect others by contract tracing, control measures could be made more efficient by targeting them to this high-risk group.

To scale up contact tracing without the massive workforce that is required for manual contact tracing, digital exposure notification apps have been developed. They rely on proximity data from smartphones or other mobile devices to identify close contacts between users. If an individual user is identified as being infectious, prior close contacts are notified and can then self-isolate. The exposure notification is deterministic (a user is only notified when potentially exposed), and it only uses nearest-neighbor information on the network of close contacts among users. Exposure notification apps have not seen widespread use, in part perhaps because of privacy concerns and early implementation challenges but likely also because of the limited binary information they provide.

### SUMMARY

Computer systems and methods are described herein to exploit the same contact information on which exposure notification applications (“apps”) rely, but that do so more effectively, thanks to a mathematical modeling framework that (1) accounts for data from varied sources, (2) spreads information to other users on the basis of calibrated scientific models of virus transmission and disease progression, and (3) spreads a richer form of information to provide a more comprehensive individual risk assessment.

Individual risks of exposure and infectiousness are sent to users by collecting crowdsourced information about infection risks and running that information through a central server that assimilates the data into a model of virus transmission and disease progression on a dynamic contact network established by proximity data from mobile devices. Periodically updated individual risks of having been exposed or of being infectious are provided, which take the place of the deterministic assessments in exposure notification apps on user devices. The systems and methods herein can be applied to any infectious disease or condition: for example, influenza, sexually transmitted diseases, Ebola virus, chickenpox, diphtheria, etc. They can also be applied to any infectious process, so long as there is a model of transmission and distributed proximity data available.

According to a first aspect of the present disclosure, a system for disease risk assessment is disclosed comprising: a server wirelessly connected to a plurality of mobile devices, with each of the plurality of mobile devices configured to provide proximity data to the server and health data related to corresponding users of the plurality of mobile devices to the server, proximity data being data that the system can use to calculate proximities between each of the plurality of mobile devices; the server being configured to: (i) build a contact network of the plurality of mobile devices based on the proximity data, (ii) assign health data collected from the plurality of mobile devices to nodes of the contact network, (iii) use an epidemiological model run forward in time over the network and in conjunction with the assigned data to produce a risk network forecast, (iv) assess individual risks of being at least one of exposed or infectious based on the risk network forecast, (v) send updated risk assessments to the plurality of mobile devices, then at a later time, (vi) receive updated proximity data and updated health data from the plurality of mobile devices; and (vii) repeat (i) through (v) at the later time.

According to a second aspect of the present disclosure a system for infectious process risk assessment is disclosed, comprising: a server wirelessly connected to a plurality of mobile devices, with each of the plurality of mobile devices configured to provide proximity data to the server and personal data related to corresponding users of the plurality of mobile devices to the server, proximity data being data that the system can use to calculate proximities between each of the plurality of mobile devices; the server being configured to: build a risk network of the plurality of mobile devices based on the proximity data, an infectious process model, and the personal data; run an ensemble of infectious process models to produce a forecast of a state of the risk network; assess individual risks of being exposed or infectious based on the forecast; receive updated proximity data and updated personal data from the plurality of mobile devices; update the risk assessment based on the updated proximity data and updated personal data; and send updated risk assessments to the plurality of mobile devices.

According to a third aspect of the present disclosure, a computer server or server network is disclosed, comprising: a processor; memory tied to the processor; the server configured to: build a risk network of a plurality of mobile devices based on proximity data, an infectious process model, and personal data; run an ensemble of infectious process models to produce a forecast of a state of the risk network; assess individual risks of being exposed or infectious based on the forecast; receive updated proximity data and updated personal data from the plurality of mobile devices; update the risk assessment based on the updated proximity data and updated personal data; and send updated risk assessments to the plurality of mobile devices.

The aspects and embodiments described above are exemplary and not comprehensive. The systems and methods can be applied to any transmission process between individuals where proximity data is collected at an individual level, and there exists underlying mathematical or data-driven models of transmission between individuals dependent on proximity. The portions of the systems and methods can be combined and implemented in any reasonable manner, not merely the ones listed above.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 shows an example of the system.

FIG. 2 shows an example of a risk network.

FIG. 3 shows an example of evolution and adjustment of the risk network.

FIG. 4 shows an example of an algorithm flow of the system.

### DETAILED DESCRIPTION

As used herein, the term “risk network” when related to refers to a mapping of individuals with their respective risk-related data and their contact connections between the individuals.

As used herein, the term “data assimilation” (or “assimilation”) refers to the combination of models of a system with data (observations) to assess the state of the system. Data assimilation as understood here is a Bayesian estimation process.

As used herein, the term “ensemble” refers to the use of multiple states of a system to produce a range of possible (past, present, or future) states. This can be seen as a form of Monte Carlo analysis.

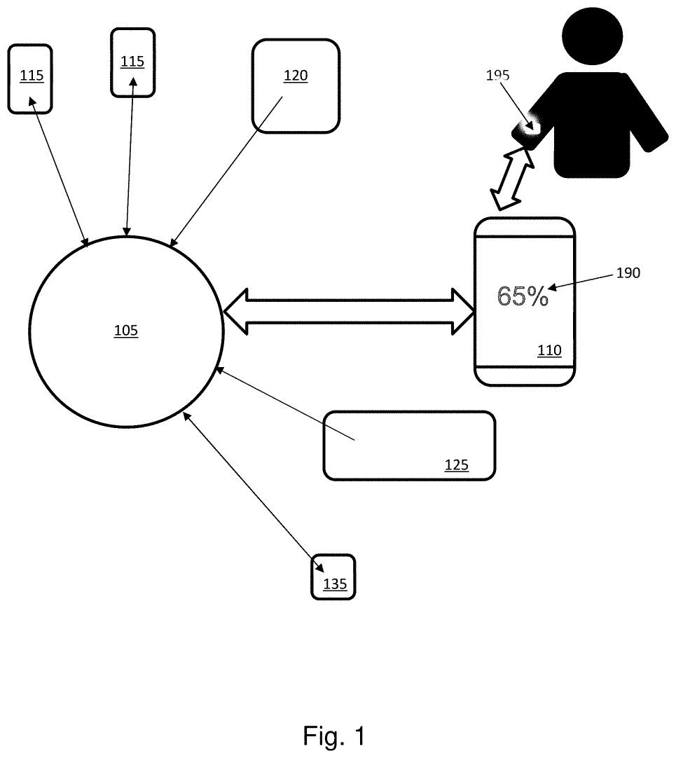

FIG. 1 shows an example of the system. A central server, server bank, or cloud of servers (105) gathers information from the user device (110), other user devices (115), computers (120), diagnostic machines (125), and/or wearable medical devices (135). Examples of user devices (110, 115) include smart phones, tablets, smart watches or other similar wearable devices, or portable computers, having some network transmission capability (e.g., 5G, WiFi, Bluetooth) and some determination system for proximity to other devices (e.g., Bluetooth) or for location (e.g., GPS, WiFi, or cellular triangulation). The computers (120) include desktop or server computers, for example ones located at a healthcare facility. The diagnostic machines (125) include medical diagnostic machines that can send data to the server (105). The wearable medical devices (135) include custom made smart tags or bracelets worn by people concerned with disease exposure (e.g., critical care workers, nurses, essential workers) that can wirelessly report proximity (either directly by proximity detection or indirectly by location determination) to other wearers to the server (105). The connections to the server (105) can be direct or indirect (e.g., through other network systems). The user device (110) is capable of displaying the user's risk probability (190) (aka “risk assessment”) to the user based on the server's (105) determination from the information gathered from the user device (110) and other devices (115, 120, 125, 135). The user device (110) can also include a connection, wired or wireless, to a biomedical sensor (195), such as a Bluetooth™ connected temperature sensor.

Risk probability can be displayed as a numerical percent chance of exposure (e.g., 55%), a bar graph (e.g., a bar that fills in more of the bar as the risk increases), a symbolic risk rating system (e.g., a number from 1 to 5, or a grading from A to D, or graphical symbols signifying risk, or a color coded system from safe-green to danger-red, etc.), or a selection of words indicating level or risk (e.g. “safe”, “low risk”, “high risk”). The number of levels of risk can be two or more, with two levels just being “safe” vs. “danger”.

In some embodiments, when device location data is available, the system can also identify “hot-spots” of high risk of transmission. By combining location data of the nodes and their individual risk assessments/states, specific regions (e.g., neighborhood, campus, base, etc.) can be given a geographic risk assessment value. This information can be sent to users so that the device can either display a level of risk for the region (similar to individual risk assessment above, but aggregated over regional groups of individuals) and/or a map with high-risk areas designated (e.g., a zone tinted red to indicate a high risk). The map can be generated at the server and be transmitted to the devices, or it can be generated at the device using risk assessment data sent from the server.

The information gathered from the various devices (110, 115, 120, 125, 135) can include some indication (direct or indirect) of proximity to the user device (110). This can be by location services, user input, static location (for non-mobile devices), or some proximity detection system.

The information gathered from the various devices (110, 115, 120, 125, 135) can include information (health and vital status data, herein “health data”) to determine the risk assessment for exposure to or being infected with the disease. This can include medical diagnostic information (e.g., body temperature, antibody counts, diagnostic test results, symptoms, medical diagnoses). In some cases, the devices can carry out the diagnostic test (e.g., temperature sensors to measure body temperature). The information can also include information related to safety precautions (e.g., vaccinations, mask wearing, time in quarantine), risk factors (e.g., pre-existing conditions, age), or other relevant data for determining risks of exposure and disease progression and spread.

The network (server+devices) learns automatically from the data and improves its risk assessments over time. This can be done with a data assimilation method, which combines models with data, such as used in weather prediction. Data assimilation adjusts a model based on the data that is gathered. For example, an ensemble adjustment Kalman filter (EAKF) can be used for data assimilation. See e.g. “An Ensemble Adjustment Kalman Filter for Data Assimilation” by Jeffrey L. Anderson (Monthly Weather Review, Vol. 129, p. 2884).

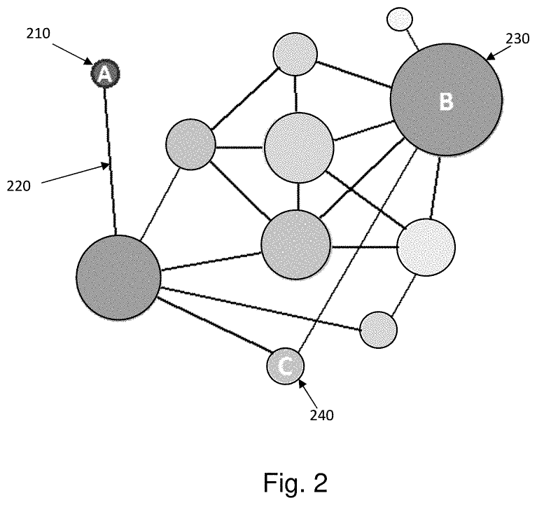

FIG. 2 shows an example schematic of a snapshot from a time-dependent risk network in which nodes (e.g., 210, 240, 230) represent individuals and edges (e.g., 220) represent close-proximity contacts between the individuals. Node A (210) is an example of a confirmed infectious individual, here indicated by the dark shade. Here, the darkness of the node indicates infection risk, with the darkest shade indicating confirmed infection. Node B (230) is an example of an individual with many contacts (edges) compared to, for example, node C (240). The size of the nodes in this example scale with the number of connections (incident edges) on that node, which is why node B (230) with six connections is larger than node C (240) with two connections, which, in turn, is larger than node A (210) with only one connection. The risk of infection generally increases with the number of connections but also depends on the infection risk of the connected nodes, and the data of the node itself (e.g., mask wearing, vaccination status, use of partitions). The risk network is time-dependent in that the connections, size, and topology change over time, with any particular schematic snapshot indicating one particular time window (e.g., one day, a four-hour period, etc.).

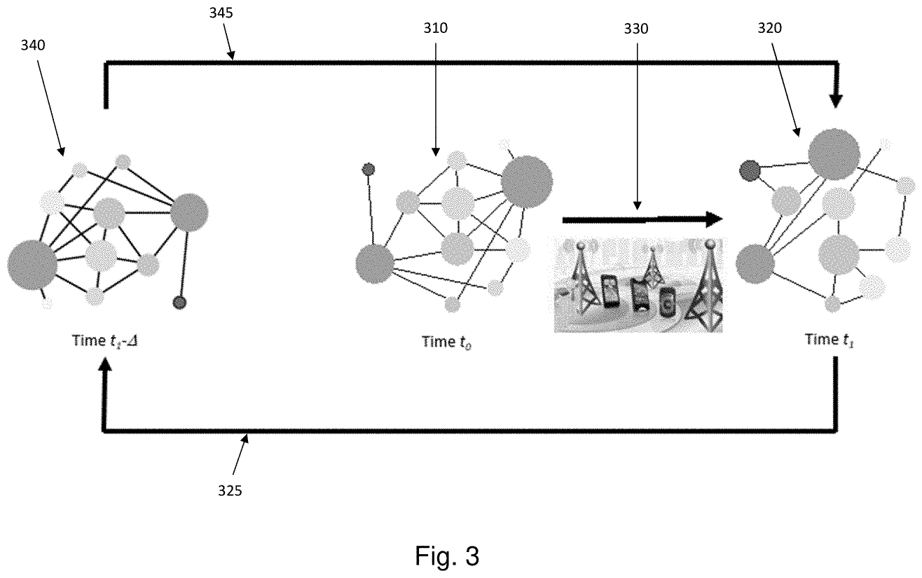

FIG. 3 shows the time-evolution of the risk network, with risk-assessment improvement through regular adjustment of the risk network state. The state of the contact network (320) at time t₁ is established from time t₀ (310) based on proximity data transmitted (330) by the devices to the server(s); health data of individuals is also received from devices (330) between times t₀ and time t_{1.} This information is then used to modify (325) the state of the risk network from a prior time t₁−Δ (340). The system is then evolved forward (345) to time t₁. This retroactive loop (325, 345) can be repeated multiple times. The retroactive updating is explained as follows: consider a node going from “susceptible” to “hospitalized” at t₁ (320). This may indicate that the same node in an earlier network (340) (e.g., a few days earlier) was incorrectly listed as “susceptible” when it should have been “infectious”. The earlier network state t₁−Δ (340) is therefore updated to address this misspecification and re-evolved (345) to produce a more accurate current network state (320), thereby creating new risk assessments for all users at t₁, which can then be pushed to their respective devices, giving the users a more accurate risk probability assessment.

The interpretation of Δ (delta) is the length of time window over which the risk network state is retroactively updated. A large value of delta can be chosen, for example, when a long history of state information is needed to provide an accurate update of the present state when new data is acquired; such settings are seen for embodiments where the infectious process model has long timescales, e.g., when virus incubation periods are long (relative to data acquisition), or if the model has strongly nonlinear dynamics, e.g., if transmissions occur with high probability for short interactions.

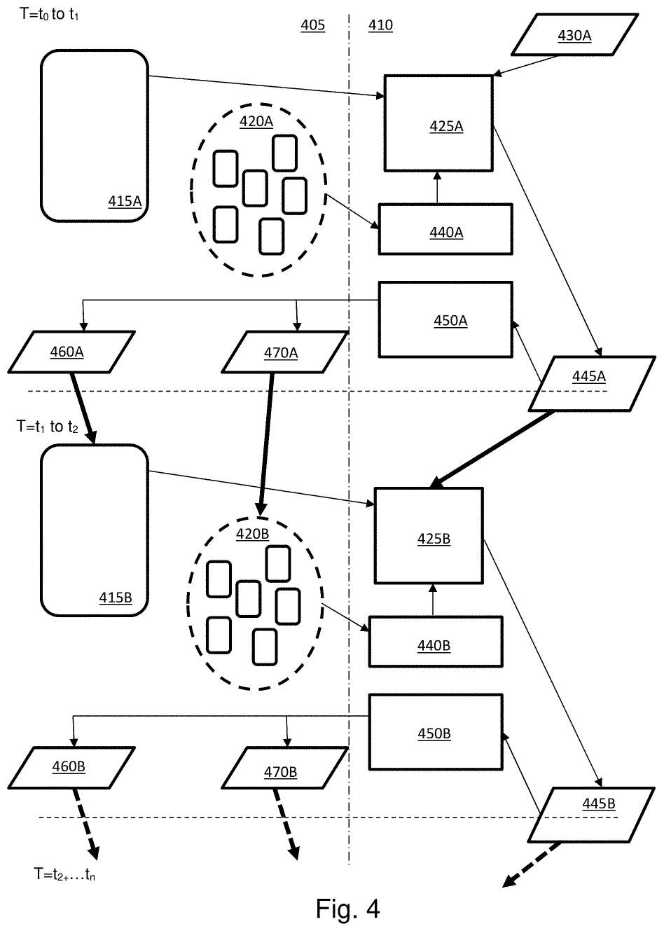

FIG. 4 shows an example of an algorithm flow of the system described herein. The system can be divided into two realms: the distributed side (405), including the various devices providing the data; and the data center side (410), including the servers performing the propagation of the disease model on the network and providing the risk assessment. Choose a positive value of Δ (as defined in the description of FIG. 3). The storage module (430A) contains a user state history, contact network history, and health data history over the time period from t₀−Δ to t₀. Over the subsequent period from t₀ to t₁=t₁+Δ, the devices provide proximity data (420A) that generates an evolving contact network (440A) as well as user-level state data (415A) based on data input (e.g., virus tests, temperature sensors, etc.). These are fed into a data assimilation module (425A) (e.g., software) together with the user states (i.e., probabilities of being infectious or exposed) at t₀ and stored history (430A) to produce data-consistent user states at t₁ that are stored in a storage module (445A) (as described using FIG. 3). The storage module (445A) also contains the data-consistent user state history, contact network history, and health data history over the time period from t₁−Δ to t₁. The user states at t₁ are then postprocessed (450A) to classify the users into infection or exposure levels. The updated states and classification results are provided to the users as personal risk assessment values (460A), with recommended user-level actions (470A) (e.g., isolating infectious users).

Collect proximity data (420B) and the user data (415B) over the time period from t₁ to t₂ (the next window) and update the contact network over the period from t₁ to t₂ (440B), and feed through the data assimilation module (425B) with the user state history, contact network history, and health data history (445A). This results in updated data-consistent user states at time t₂ stored in a storage module (445B). The storage module (445B) also contains a user state history, contact network history, and health data history over the time period from t₂−Δ to t₂. They are again postprocessed (450B) to classify users into infection or exposure levels, and the results are provided to the users (460B and 470B). The cycle (415B, 420B, 440B, 425B, 445B, 450B, 460B, 470B) then repeats for the windows between consecutive times t₂, . . . , tₙ.

In some embodiments, each node of the risk network represents an individual and its risk status according to an epidemiological disease model, and the time-dependent connections between nodes represent temporary contacts between individuals during which an infectious transmission process can occur.

An example epidemiology model is the SEIHRD model (Susceptible-Exposed-Infectious-Hospitalized-Removed-Deceased) or its variants. The SEIHRD models a population of N individuals i (with i=1, . . . , N). At any time t, an individual i is in exactly one of 6 health and vital states:
Sᵢ(t)=Susceptible, when they can get infected with the virus;
Eᵢ(t)=Exposed, when infected with the virus but not yet infectious;
Iᵢ(t)=Infectious, when shedding the virus (with or without clinical symptoms) but not hospitalized;
Hᵢ(t)=Hospitalized, when hospitalized with active disease, in which case individuals are assumed to be shedding the virus (if not, this can be taken into account by modifying their individual virus transmission rates);
Rᵢ(t)=Resistant when immune to the disease through either vaccination or immunity conferred by a prior infection; or
Dᵢ(t)=Deceased.

The states Sᵢ(t), Eᵢ(t), Iᵢ(t), Hᵢ(t), Rᵢ(t), and Dᵢ(t) can be taken as Bernoulli random variables that depend on the time (t) and only take the values of 0 and 1. For example, Sᵢ(t)=1 when individual i is susceptible at time t, and not susceptible when Sᵢ(t)=0 (likewise for the other state variables). Since the variable describe all the possible states of a device user and since they are considered exclusive of each other, Sᵢ+Eᵢ+Iᵢ+Hᵢ+Rᵢ+Dᵢ=1.

Each individual is represented by a node on the time-dependent network (see, e.g., FIGS. 2 and 3), with time-dependent edges between nodes established by close contacts. The virus is transmitted across active edges from infectious or hospitalized nodes to susceptible nodes, which become exposed when transmission occurs. The probability of transmission increases with contact duration, and the transmission rate can vary from node to node and with time, for example, to reflect a reduced transmission rate when personal protective equipment (PPE) is worn. From being exposed, nodes progress to becoming infectious, and later they either recover and become resistant, progress to requiring hospitalization, or die. Hospitalized nodes, in turn, either recover and become resistant, or they die. The transition rates between the health and vital states of each node can vary from node to node. For example, disease progression varies individually depending on age and medical risk factors in ways that the platform described herein can learn from.

### Example SEIHRD

Transmission along the temporary edges from one node to another and transitions between health and vital states within each node are modeled as independent Poisson processes. Each process is characterized by a rate that may vary from node to node and may depend on external variables such as age, sex, and medical risk factors.

The following assumptions about the transmission rate and the parameters characterizing transition rates between SEIHRD states, including prior distributions used in the network model for DA can be made:

1) Transmission rate: During the contact period between an infectious or hospitalized individual (Iⱼ(t)=1 or Hⱼ(t)=1; j being the node connected to node i) and a susceptible individual (Sᵢ(t)=1), virus can be transmitted across the edge between nodes j and i. When transmission occurs, the susceptible node i becomes exposed and switches state to Eᵢ(t)=1. During the contact period in which an edge is active (wⱼᵢ(t)=1), assume the transmission rate from an infectious node with Iⱼ(t)=1 to a susceptible node with Sᵢ(t)=1 is κ^{I}ⱼᵢ=aⱼᵢ(t)β, and that from a hospitalized node with Hⱼ(t)=1 is κ^{H}ⱼᵢ=a′ⱼᵢ(t)β. The parameter β is a transmission rate across active edges, which data assimilation can learn as a global (constant for all nodes), group (constant for multiple nodes), or individual (different for each node) parameter. The time dependent functions aⱼᵢ and a′ⱼᵢ are transmission rate modifiers that can be adjusted to incorporate additional information that may be available—for example, user-supplied information that individual i is using PPE at time t. Examples include using aⱼᵢ(t)=0.1 within hospitals and aⱼᵢ(t)=1 otherwise, and a transmission rate β=0.5 h⁻¹=12 day⁻¹ for a respiratory virus. Modeling the transmission as a Poisson process, the probability that transmission occurs during contact increases with the duration of the contact period τ, e.g., for an infectious node as Tⱼᵢ(τ)=1−exp(−κ^{I}ⱼᵢτ). This holds, provided that the contact period τ is short relative to the duration of infectiousness, so that the infectiousness status of a node does not change during contact.

2) Latent period: Exposed nodes with Eᵢt)=1 transition to being infectious with Iᵢ(t)=1 at the rate σᵢ, which is the inverse of the latent period: the time it takes for an exposed individual to become infectious. For example, for COVID-19, the latent period lies between about 2 days and about 12 days. The latent period σᵢ⁻¹ can be taken to be fixed for each node i but heterogeneous across nodes; it too can be learned by data assimilation.

3) Duration of infectiousness in community: Infectious nodes with Iᵢ(t)=1 transition to resistant (R), hospitalized (H), or deceased (D) at the rate γᵢ, which is the inverse of the duration of infectiousness in the community (i.e., outside hospitals Like σᵢ, γᵢ can be taken to be fixed for each node i but heterogeneous across nodes and can be learned by data assimilation.

4) Hospitalization rate: Assume a fraction hᵢ of infectious nodes with Iᵢ(t)=1 requires hospitalization after becoming infectious. More precisely, we assume that infectious nodes transition to becoming hospitalized at the rate hᵢγᵢ. This implies that, over a period Δt that is short relative to the duration of infectiousness γᵢ⁻¹, the probability of transitioning from being infectious to hospitalized, relative to the total probability of leaving the infectious state, is

$$\frac{1 - {e}^{- {h}_{i j} Δ t}}{1 - {e}^{- {y}_{i j} Δ t}} \approx {h}_{i} for {γ}_{i} Δ t ⪡ 1$$

The parameter hᵢ can be taken to be fixed for each node i but heterogeneous across nodes; it generally depends on age and other risk factors and can be learned by data assimilation.

5) Mortality rate: Assume a fraction dᵢ of infectious nodes with Iᵢ(t)=1 and a fraction d′_{I} of hospitalized nodes with Hᵢ(t)=1 die.

6) Resistance: Resistance can be assumed to be lifelong or temporary, so that an individual (node) who becomes resistant remains so indefinitely or returns to being susceptible over some time, depending on the assumption.

The health and vital states and transition rates define a Markov chain for the individual-level SEIHRD states. The SEIHRD Markov chain on a contact network can be simulated directly with kinetic Monte Carlo methods. Kinetic Monte Carlo simulations can be used both to benchmark a model for the SEIHRD probabilities and to provide a surrogate for the real world for simulations.

### Reduced Master Equations

The individual SEIHRD probabilities are the expected values, <Sᵢ(t)>, <Eᵢ(t)>, etc. associated with the Bernoulli random variables for the states. That is, <Sᵢ(t)> is the probability that individual i is susceptible at time t.

These probabilities could be obtained as averages over an ensemble of kinetic Monte Carlo simulations; however, it is more computationally efficient to solve reduced master equations for the probabilities directly.

In the reduced master equations for the probabilities <Sᵢ(t)>, <Eᵢ(t)>, etc., one can include an exogenous infection rate η. This allows for infection from outside the network of Ñ users when the user network represents only a subset of a larger network with N nodes, and so transmission can occur from unaccounted nodes. The exogenous infection rate can be scaled by the number of external neighbors kᵢ^{x} of node i that are not part of the user network; thus, a user surrounded by other users will have no exogenous infection rate, while users with many external neighbors will have a larger exogenous infection rate.

### Closure of Reduced Master Equations

The master equations for the probabilities are not closed because they depend on the joint probabilities <Sᵢ(t)Iⱼ(t)> and <Sᵢ(t)Hⱼ(t)>. Different closed form expressions may be used. The simplest closure, the mean-field approximation, where <Sᵢ(t)Iⱼ(t)>=<Sᵢ(t)><Iⱼ(t)>, and <Sᵢ(t)Hⱼ(t)>=<Sᵢ(t)><Hⱼ(t)>, is often accurate for real-world networks and can be used.

### Data Assimilation Algorithm

For data assimilation, a version of the ensemble adjustment Kalman filter (EAKF) can be used. EAKF treats an ensemble of M model parameters and states S^{m}(t), E^{m}(t), etc. from a previous data assimilation cycle as a prior and then linearly updates the ensemble of model parameters and states to obtain a risk forecast (e.g., an approximate Bayesian posterior) on states of the risk network, it makes no assumptions about the network structure and it scales well to high-dimensional problems.

The risk forecast provides a prediction of the epidemiological state of the users at the current time (accounting for the history of proximity data and health data). The state describes the probability of any user being in a particular category (S, E, I, H, R, D), in particular the E and I category are of interest of describing the risk of being exposed or the risk of being infectious. A binary classification can be used to determine who is infectious given the state; define a threshold (C), if the forecasted infectiousness>C then consider the user infected, else do not. To choose a value for the threshold one can look at performance metrics, such as Receiver Operator Characteristic curves—which demonstrate the efficiency of this classification (e.g., for a given value of C, how many users are categorized as infectious in the population vs how many users were correctly identify as infectious). There are different ways of choosing optimal values from this analysis. It may be of interest to use the E category to classify users as exposed, or one could use a combination of E and I to classify exposed and infectious users.

One could convert the probability into different forms of classification rather than just a binary classifier (e.g., one could represent the probability as a percentage, or using multiple classification values). The optimal threshold value of these classifiers can change over time. Thus, one can also try to choose a variable threshold that is calculated online. One could use other criteria for choosing the threshold.

The models can be epidemiological models for modeling disease, or more generally infectious process models for modeling any infectious process. The models are capable of evolving the risk of infectiousness, of any individual in the network forward in time, and are capable of evolving the risk of infection via transmission between nodes on the risk network, dependent on the network structure.

### Parameter Learning

In addition to assimilating probabilities of SEIHRD states, one can in principle learn about parameters in the reduced master equation model from data, as examples:
Individual partial and time-dependent transmission rates βᵢ; where transmission rate between node I and j is given by β=0.5(βᵢ+βⱼ),
Individual inverse latent periods σᵢ;
Individual inverse durations of infectiousness γᵢ and hospitalization γᵢ′;
Individual hospitalization rates hi and mortality rates dᵢ and dᵢ′.

### Postprocessing: Classification of Infected Users

Nodes i in the community group (c) can be classified as possibly infectious (Iᵢ=1) or not (Iᵢ=0) according to (Iᵢ=1 if <Iᵢ^{m}>>c_{I}, 0 otherwise). Here, c_{I} is a classification threshold, which can be determined from receiver operating characteristic (ROC) curves as some optimum tradeoff between wanting to achieve high true positive rates while keeping false positive rates modest. The ROC curves used are adapted to the setting in which the prevalence of infectiousness is relatively low and what is normally of interest is the fraction of users that is classified as possibly infectious (and thus may be asked to self-isolate). The true positive rate (TPR, nodes with Iᵢ=1 for which Iᵢ=1 in the stochastic simulation) can be plotted against the predicted positive fraction (PPF, fraction of nodes with Iᵢ=1 in the user base of size Ñ), where these statistics are available (e.g., in stochastic network simulations to benchmark the classification thresholds). ROC curves are traced out by lowering the classification threshold c_{I}, thereby increasing both TPR and PPF.

Intervention Example: Lockdown vs. Isolation

### a) Specification of Simulated Population

Intervention scenarios can be tested on simulated infectious disease through a simulated population. An evidenced example is a simulated population of N=97,942 individuals, and 1 million connections between them. They are provided with a five-category age distribution consistent with New York City data. The population has a similar degree distribution to that of human networks. A member of the population is either a community member (c), hospitalized patient (h) or healthcare worker (w); group (h) is connected only to group (w); initially group (c) contains 95% of the population and group (w) contains 5%. The human network degree distribution is based on social-contact analyses and uses a power-law degree correction for group (c) with exponent 2.5, and mean degree 10 and maximum degree is 100; the connections for groups (h) and (w) use an Erdös-Rényi model with mean degree 5 for group (h) and 10 for group (w), and a mean degree of 5 for contacts between the groups.

The time evolution of contacts between individuals is stochastic and governed by a law that follows a daily cycle with minimum contact rate at midnight λₘᵢₙ=4 day⁻¹, and maximum contact rate at midday λₘₐₓ=84 day⁻¹. An example stochastic process is a birth-death process, with mean rate Aⱼᵢ(t),

$${A}_{j i} ( t ) = \frac{1}{\hat{k}} max \{ min ( {λ}_{j , min} , {λ}_{i , min} ) , min ( {λ}_{j , max} , {λ}_{i , max} )$$

Here \grave{k}=10 is the mean degree of the community network group, t=0 starts at midnight with units of days, and λ_{i, min}, λ_{i, max} refer to an individual's contact rate minimum and maximum. The contact durations are exponentially distributed with mean contact duration τ=2, calibrated to high-resolution human contact data.

If a node becomes hospitalized it is deactivated at its previous location in the network and transferred to the hospital group (h). The hospital has no capacity restrictions (though one could be imposed).

### b) Specification of Simulated Epidemic

The simulated epidemic is for COVID-19, using the example SEIHRD model, and parameters within.

### c) The Lockdown Scenario

In the first, an example lockdown scenario, set λ_{i, max} for all nodes in the community group (c) to 33 day⁻¹. This amounts to a reduction of the mean contact rate in group (c) by 58%.

### d) The Targeted Intervention Scenario

In the second scenario, an example time-limited isolation intervention, targets reduction of the contact rates of high-risk nodes, as determined by the postprocessing classifier of the described system, by setting λ_{i, max}=λ_{i, min}=4 day⁻¹; thus, these high-risk nodes are assumed to self-isolate, with only 4 contacts per day on average, corresponding to a reduction of their average contact rate by 91%. This continues for a period of 7 days, after which the contact rates are reset to their original values, if the individuals are no longer high risk.

To determine high risk nodes, 100% of the population are taken to be users of the system (a smaller percentage can be used). Of this user population, health data is provided by a random 5% once per day (other state-dependent strategies can be used), in the form of results from rapid diagnostic tests with accuracy specified by the sensitivity (80%) and specificity (99%), consistent with current rapid test accuracy for COVID-19. This data, along with previous user states given by the reduced master equations, are given to the data assimilation algorithm, and then states are postprocessed with an isolation threshold of c_{I}=1% (a time-dependent classification can also be used) to determine a binary isolation guideline for each user (“isolate” or “do not isolate”). We assume compliance by the users to this guideline.

### e) Comparison of Scenarios

The scenarios were run for 120 days, and with no intervention, the peak of the simulated epidemic would occur at approximately 30 days; the interventions were both initiated at 10 days.

The targeted intervention scenario suppresses the epidemic more effectively than the lockdown scenario, and cumulative deaths are reduced by 50-70% overall. The lockdown scenario requires 100% of the population to have reduced contacts, whereas the targeted intervention scenario allowed 83-85% of users to have no reduction in contacts during the first 7 days of intervention, rising quickly to 90-95% after this; of those self-isolating, 50% leave isolation within 7 days, and 90% within 14 days.

A number of embodiments of the disclosure have been described. Nevertheless, it will be understood that various modifications may be made without departing from the spirit and scope of the present disclosure. Accordingly, other embodiments are within the scope of the following claims.

The examples set forth above are provided to those of ordinary skill in the art as a complete disclosure and description of how to make and use the embodiments of the disclosure, and are not intended to limit the scope of what the inventor/inventors regard as their disclosure.

Modifications of the above-described modes for carrying out the methods and systems herein disclosed that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

It is to be understood that the disclosure is not limited to particular methods or systems, which can, of course, vary. It is also to be understood that the terminology used herein is for the purpose of describing particular embodiments only and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

1. A system for infectious disease risk assessment comprising:
a server wirelessly connected to a plurality of mobile devices, with each of the plurality of mobile devices configured to provide proximity data to the server and health data related to corresponding users of the plurality of mobile devices to the server, proximity data being data that the system can use to calculate proximities between each of the plurality of mobile devices;
the server being configured to:
(i) build a contact network of the plurality of mobile devices based on the proximity data,
(ii) assign health data collected from the plurality of mobile devices to nodes of the contact network,
(iii) use an epidemiological model run forward in time over the network and in conjunction with the assigned data to produce a risk network forecast,
(iv) assess individual risks of being at least one of exposed or infectious based on the risk network forecast,
(v) send updated risk assessments to the plurality of mobile devices, then at a later time,
(vi) receive updated proximity data and updated health data from the plurality of mobile devices; and
(vii) repeat (i) through (v) at the later time.

2. The system of claim 1, wherein the server is part of a plurality of servers working cooperatively.

3. The system of claim 1, wherein the plurality of mobile devices comprises one or more of: smartphones, tablets, and wearable computers.

4. The system of claim 1, wherein the contact network consists of nodes representing users of the plurality of mobile devices and edges representing contacts between the users.

5. The system of claim 4 wherein the nodes include infection status of each nodes' corresponding user.

6. The system of claim 1, wherein the forecast includes data assimilation.

7. The system of claim 6, wherein the data assimilation includes ensemble processing.

8. The system of claim 7, wherein the ensemble processing includes an ensemble Kalman filter.

9. The system of claim 1, wherein at least one of the plurality of mobile devices includes a temperature sensor configured to measure body temperature, and wherein the at least one of the plurality of devices includes the body temperature in the health data.

10. The system of claim 1, wherein the health data includes at least one of: body temperature, symptoms, medical diagnosis, vaccinations, pre-existing conditions, age, mask wearing, virus test results, and antibody count.

11. The system of claim 1, wherein the risk assessment comprises a probability of individual exposure.

12. The system of claim 1, wherein the risk assessment comprises heat map information indicating high risk areas.

13. The system of claim 1, wherein the proximity data includes location services data.

14. A system for infectious process risk assessment comprising:
a server wirelessly connected to a plurality of mobile devices, with each of the plurality of mobile devices configured to provide proximity data to the server and personal data related to corresponding users of the plurality of mobile devices to the server, proximity data being data that the system can use to calculate proximities between each of the plurality of mobile devices;
the server being configured to:
build a risk network of the plurality of mobile devices based on the proximity data, an infectious process model, and the personal data;
run an ensemble of infectious process models to produce a forecast of a state of the risk network;
assess individual risks of being exposed or infectious based on the forecast;
receive updated proximity data and updated personal data from the plurality of mobile devices;
update the risk assessment based on the updated proximity data and updated personal data; and
send updated risk assessments to the plurality of mobile devices.

15. The system of claim 14, wherein the server is part of a plurality of servers working cooperatively.

16. The system of claim 14, wherein the plurality of mobile devices comprises one or more of: smartphones, tablets, and wearable computers.

17. The system of claim 14, wherein the contact network consists of nodes representing users of the plurality of mobile devices and edges representing contacts between the users.

18. The system of claim 17 wherein the nodes include infection status of each nodes' corresponding user.

19. The system of claim 14, wherein the forecast includes data assimilation.

20. The system of claim 19, wherein the data assimilation includes ensemble processing.

21. The system of claim 20, wherein the ensemble processing includes an ensemble Kalman filter.

22. The system of claim 14, wherein the risk assessment comprises a probability of individual exposure.

23. The system of claim 14, wherein the risk assessment comprises heat map information indicating high risk areas.

24. The system of claim 14, wherein the proximity data includes location services data.

25. A computer server or server network, comprising:
a processor; and
memory tied to the processor;
the server configured to:
build a risk network of a plurality of mobile devices based on proximity data, an infectious process model, and personal data;
run an ensemble of infectious process models to produce a forecast of a state of the risk network;
assess individual risks of being exposed or infectious based on the forecast;
receive updated proximity data and updated personal data from the plurality of mobile devices;
update the risk assessment based on the updated proximity data and updated personal data; and
send updated risk assessments to the plurality of mobile devices.
